(19) United States
(12) Patent Application Publication
(10) Pub. No.: US 2026/0066095 A1
(43) Pub. Date: Mar. 5, 2026

(54) Augmented Reality for Medical Test and Display

(71) Applicant: Star Solutions International Inc., Richmond (CA)

(72) Inventor: Yichuang Jin, Vancouver (CA)

(21) Appl. No.: 18/817,841

(22) Filed: Aug. 28, 2024

(51) Int. Cl. G16H 30/40; G16H 40/63

(52) U.S. Cl. CPC G16H 30/40; G16H 40/63

(57) ABSTRACT

An embodiment may include an example method, which may involve causing an imaging device to capture an image representation of an organism, wherein the image representation includes at least part of a body of the organism, determining, within the image representation, an expected location of a physical organ within the body, causing a graphical interface to display a virtual organ model superimposed upon the image representation at the expected location of the physical organ, wherein the virtual organ model is a digital replica of physical characteristics of the physical organ, and causing the graphical interface to display a marker on the virtual organ model representing a location of where a medical device is to be positioned with respect to the body.

### BACKGROUND

Due to advancements in technology, many types of professional medical equipment, including electrocardiogramhoscopes, and handheld ultrasound scanners, can be obtained by the public. However, due to the complexity of these types of equipment, generally only medical professionals are able to properly use such equipment and correctly interpret their output.

### SUMMARY

Current approaches to the use of medical technologies are limited by the complexity of medical equipment, which often produce large amounts of data and are difficult for the general public to use. This complexity may cause users to spend more time navigating and refreshing pages, menus, and other components of a graphical interface used to operate the medical equipment, thereby using more computational and memory resources.

In contrast, the embodiments herein involve a streamlined approach to the use of medical devices, creating an intuitive process for positioning medical devices on bodies, and collecting output related to the functioning of organs. Such data can be used to improve the positioning process. This then saves computational and memory resources that would be otherwise spent by users taking time navigating and refreshing pages, menus, and other components to operate complex medical equipment. Additionally, the embodiments herein allow users to take medical readings without the need to leave their home or consult with a medical professional, thus saving users' time.

Accordingly, a first example embodiment may involve causing an imaging device to capture an image representation of an organism, wherein the image representation includes at least part of a body of the organism. The first example embodiment may also involve determining, within the image representation, an expected location of a physical organ within the body. The first example embodiment may also involve causing a graphical interface to display a virtual organ model superimposed upon the image representation at the expected location of the physical organ, wherein the virtual organ model is a digital replica of physical characteristics of the physical organ. The first example embodiment may also involve causing the graphical interface to display a marker on the virtual organ model representing a location of where a medical device is to be positioned with respect to the body.

A second example embodiment may involve receiving an indication that a medical device has been positioned at a location with respect to a body of an organism, wherein the location is displayed on a graphical interface, and wherein the location was determined at least in part by a virtual organ model superimposed upon an image representation of an expected location of a physical organ of the organism. The second example embodiment may also involve, in response to receiving the indication, causing an imaging device to capture a stream of further image representations of the organism and causing the medical device to obtain measurements relating to the physical organ. The second example embodiment may also involve causing at least a portion of the stream of further image representations and a portion of the measurements to be stored in a memory, each with associated timestamps.

A third example embodiment may involve a computing system. The computing system may include at least one processor, as well as memory and program instructions. The program instructions may be stored in the memory, and upon execution by the at least one processor, cause the computing system to perform operations in accordance with the first and/or second example embodiments.

A fourth example embodiment may involve a system that may include various means for carrying out each of the operations of the first and/or second example embodiments.

A fifth example embodiment may involve a non-transitory computer-readable medium, having stored thereon program instructions that, upon execution by a computing system, cause the computing system to perform operations in accordance with the first and/or second example embodiments.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE FIGURES

FIG. 1 depicts a computing device, in accordance with example embodiments.

FIG. 2 depicts a test system, in accordance with example embodiments.

FIG. 3 depicts a flow chart of a process, in accordance with example embodiments.

FIG. 4A depicts a mobile device in a first state, in accordance with example embodiments.

FIG. 4B depicts a mobile device in a second state, in accordance with example embodiments.

FIG. 4C depicts a mobile device in a third state, in accordance with example embodiments.

FIG. 4D depicts a mobile device in a fourth state, in accordance with example embodiments.

FIG. 5 depicts a flow chart, in accordance with example embodiments.

FIG. 6 depicts a flow chart, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of software features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

Unless clearly indicated otherwise herein, the term “or” is to be interpreted as the inclusive disjunction. For example, the phrase “A, B, or C” is true if any one or more of the arguments A, B, C are true, and is only false if all of A, B, and C are false.

### I. Overview

As noted above, professional medical equipment has become more accessible, but proper use of such equipment and interpretation of the results that they produce has remained out of the reach of all but trained medical professionals. The embodiments herein overcome this problem by making use of artificial intelligence (AI) and augmented reality (AR) technologies to instruct individuals to use medical equipment in real time and to display measurements.

Professional medical equipment measurements are generally very sensitive to the positions where the equipment (or sensors of the equipment) are placed on a patient's body. For example, a stethoscope is used to measure the sound produced by valve closing inside the heart, so the diaphragm (i.e., chestpiece) of the stethoscope is generally placed closest to the heart's auscultation points (i.e. locations where it is easier to hear the heart's rhythms). As another example, a 12-lead ECG is used to measure heart repolarization and depolarization along the axis between the leads. Medical professionals have established conventions where these leads should be put and where the axes are located. However, any misplacement of these leads may produce incorrect results that could decrease the accuracy of diagnoses or other conclusions drawn from these results.

The embodiments herein may be operable with a variety of medical devices, including but not limited to: digital stethoscopes, digital thermometers, blood pressure monitors, pulse oximeters, glucose meters, ECG monitors, ultrasound machines, spirometers, fetal Doppler devices, and so on. Other examples of medical devices include an x-ray device, an ultrasound device, a magnetic resonance imaging (MRI) machine, a light detection and ranging (LiDAR) device, and/or others.

The embodiments herein may take advantage of mobile device technology (e.g., mobile phones, smartphones, and tablets) to aid the use of medical equipment. Many modern mobile devices are equipped with high-resolution cameras which can capture an image or image stream of a subject. An image stream may be captured at a high frame rate (e.g., 15-240 frames per second). From the imagery, the embodiments herein then detect the body pose (e.g., specific alignments and positions of a body and/or its parts at a given moment, possibly including orientation, angles, and placements of a head, torso, limbs, and so on). As a part of detecting the body pose, the embodiments herein may identify one or more predetermined body points. For example, the embodiments herein may identify the shoulders and hips when detecting the placement and orientation of a subject's torso. Once the body pose is detected, a digital replica of physical characteristics of a physical organ, herein a virtual organ model, is overlaid with the captured images at the expected location of the physical organ according to the detected body pose and displayed to augment the displayed image representation. For example, a heart model may be overlaid on a subject's torso, and scaled appropriately to fit the subject.

When the body pose is detected and the initial virtual organ model is placed, one or more testing points (i.e. a location of where a medical device is to be positioned with respect to the body) are established accordingly. Such testing points may vary based upon the medical device being used and the virtual organ model that has been placed. For example, for a digital stethoscope with a heart virtual organ model, a testing point may be placed around the breastbone, while for a blood pressure monitor such as a cuff, a testing point may be placed around the upper arm. These testing points are then highlighted in the images, which provides real-time instruction to the user how and where to place one or more medical devices and/or parts of medical devices with respect to the expected location of the physical organ.

The medical device may be time-synchronized to the mobile device. In other words, clocks on both the medical device and the mobile device may be synchronized. This is so that data collected by the medical device (i.e. the measurements) are properly associated with the mobile device's image stream. To accomplish this, the measurements may be timestamped according to the synchronized time, such that the results are associated with a specific moment in the image stream. Thus, when others (such as medical professionals) view the measurements, they may also be able see how the measurements were produced. The timestamped measurements may also be used to produce or calculate additional information. One example is the PTT (Pulse Transmission Time) in the case of cardiovascular applications, as PTT can be used for measuring the stiffness of blood vessels.

The measurements may also be used to further fine-tune or improve the virtual organ model. For example, in the case of a heart analysis, a captured heartbeat rate may be used to align the contracting of the heart's atria and ventricles. The heartbeat sound may also be used to align the closure of the mitral and tricuspid valves. This information may be used to improve the virtual organ model and thus improve the functioning of the system in the future.

### II. Example Systems

The following description and accompanying drawings will elucidate features of various example embodiments. The embodiments provided are by way of example, and are not intended to be limiting. As such, the dimensions of the drawings are not necessarily to scale.

In the following, “coupled to” may refer to an operable connection between two devices, such as an electrical connection or other wired connection, a wireless connection, a connection over a local or wide-area network such as the Internet, or other type of connection allowing for communication between two devices.

### A. Example Computing Devices

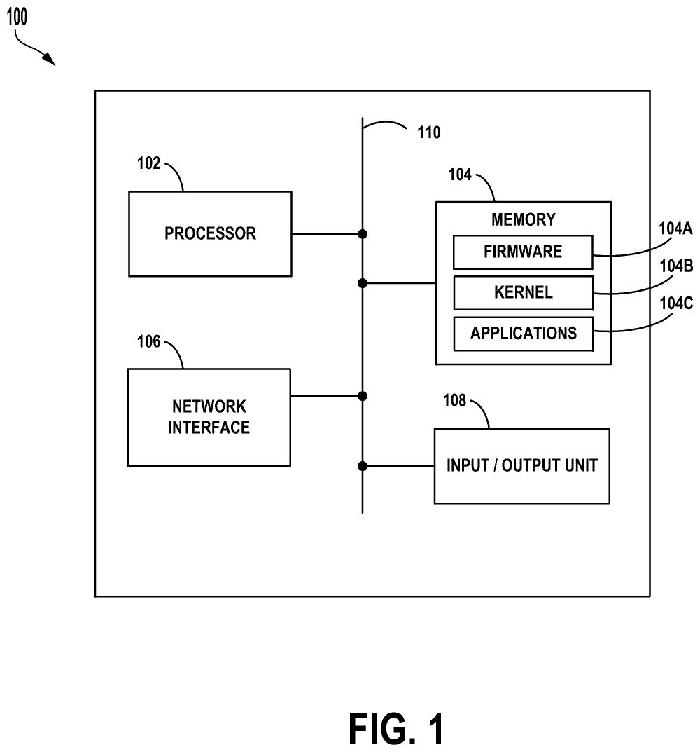

FIG. 1 is a simplified block diagram exemplifying a computing device 100, illustrating some of the components that could be included in a computing device arranged to operate in accordance with the embodiments herein. Computing device 100 could be a client device (e.g., a device actively operated by a user), a server device (e.g., a device that provides computational services to client devices), or some other type of computational platform. Some server devices may operate as client devices from time to time in order to perform particular operations, and some client devices may incorporate server features.

In this example, computing device 100 includes processor 102, memory 104, network interface 106, and input/output unit 108, all of which may be coupled by system bus 110 or a similar mechanism. In some embodiments, computing device 100 may include other components and/or peripheral devices (e.g., detachable storage, printers, and so on).

Processor 102 may be one or more of any type of computer processing element, such as a central processing unit (CPU), a graphical processing unit (GPU), another form of co-processor (e.g., a mathematics or encryption co-processor), a digital signal processor (DSP), a network processor, and/or a form of integrated circuit or controller that performs processor operations. In some cases, processor 102 may be one or more single-core processors. In other cases, processor 102 may be one or more multi-core processors with multiple independent processing units. Processor 102 may also include register memory for temporarily storing instructions being executed and related data, as well as cache memory for temporarily storing recently-used instructions and data.

Memory 104 may be any form of computer-usable memory, including but not limited to random access memory (RAM), read-only memory (ROM), and non-volatile memory (e.g., flash memory, hard disk drives, solid state drives, compact discs (CDs), digital video discs (DVDs), and/or tape storage). Thus, memory 104 represents both main memory units, as well as long-term storage.

Memory 104 may store program instructions and/or data on which program instructions may operate. By way of example, memory 104 may store these program instructions on a non-transitory, computer-readable medium, such that the instructions are executable by processor 102 to carry out any of the methods, processes, or operations disclosed in this specification or the accompanying drawings.

As shown in FIG. 1, memory 104 may include firmware 104A, kernel 104B, and/or applications 104C. Firmware 104A may be program code used to boot or otherwise initiate some or all of computing device 100. Kernel 104B may be an operating system, including modules for memory management, scheduling and management of processes, input/output, and communication. Kernel 104B may also include device drivers that allow the operating system to communicate with the hardware modules (e.g., memory units, networking interfaces, ports, and buses) of computing device 100. Applications 104C may be one or more user-space software programs, such as web browsers or email clients, as well as any software libraries used by these programs. Memory 104 may also store data used by these and other programs and applications.

Network interface 106 may take the form of one or more wireline interfaces, such as Ethernet (e.g., Fast Ethernet, Gigabit Ethernet, 10 Gigabit Ethernet, Ethernet over fiber, and so on). Network interface 106 may also support communication over one or more non-Ethernet media, such as coaxial cables or power lines, or over wide-area media, such as Synchronous Optical Networking (SONET), Data Over Cable Service Interface Specification (DOCSIS), or digital subscriber line (DSL) technologies. Network interface 106 may additionally take the form of one or more wireless interfaces, such as IEEE 802.11 (Wifi), BLUETOOTH®, global positioning system (GPS), or a wide-area wireless interface. However, other forms of physical layer interfaces and other types of standard or proprietary communication protocols may be used over network interface 106. Furthermore, network interface 106 may comprise multiple physical interfaces. For instance, some embodiments of computing device 100 may include Ethernet, BLUETOOTH®, and Wifi interfaces.

Input/output unit 108 may facilitate user and peripheral device interaction with computing device 100. Input/output unit 108 may include one or more types of input devices, such as a keyboard, a mouse, a touch screen, and so on. Similarly, input/output unit 108 may include one or more types of output devices, such as a screen, monitor, printer, and/or one or more light emitting diodes (LEDs). Additionally or alternatively, computing device 100 may communicate with other devices using a universal serial bus (USB) or high-definition multimedia interface (HDMI) port interface, for example.

In some embodiments, input/output unit 108 may include or be coupled to an imaging device. The imaging device may be a camera, an infrared camera, an x-ray device, an ultrasound device, a magnetic resonance imaging (MRI) machine, a light detection and ranging (LiDAR) device, and/or others.

In some embodiments, one or more computing devices like computing device 100 may be deployed. The exact physical location, connectivity, and configuration of these computing devices may be unknown and/or unimportant to client devices.

In some embodiments, computing device 100 may be a mobile device, such as a smartphone, other mobile phone, or tablet computer.

### B. Example Test Systems

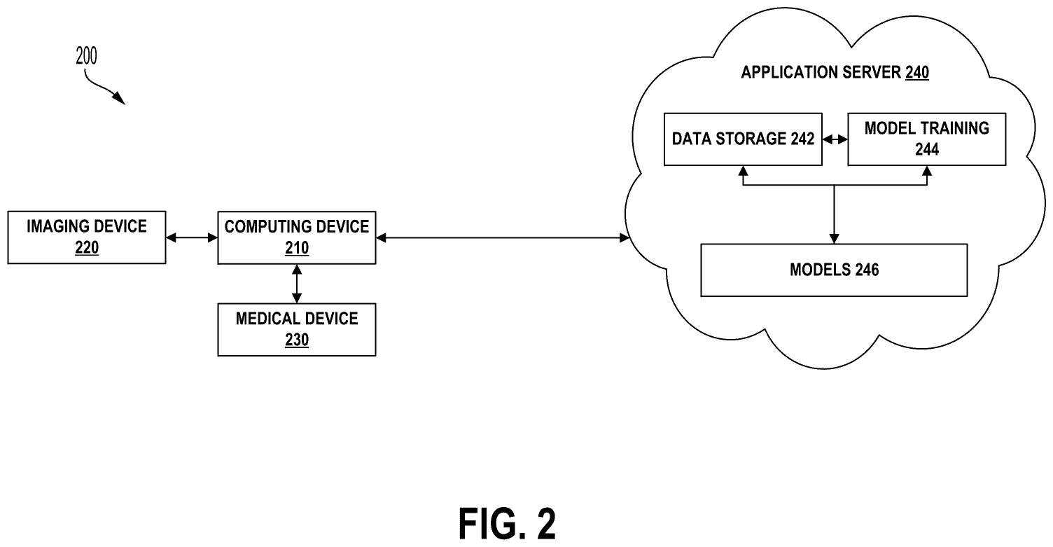

FIG. 2 illustrates an overview of an example test system 200. The test system 200 may include a computing device 210, an imaging device 220, medical device 230, and/or an application server 240.

Computing device 210 may be a computing device 100, as described above. Computing device 210 may include or be coupled to an imaging device 220. While computing device 210 and imaging device 220 are depicted as separate in FIG. 2, in some embodiments the two may be components of the same device.

Computing device 210, as noted above, may include memory 104, which may store data related to applications running on the computing device 210. In some embodiments, one or more virtual organ models may be stored in memory 104. “Virtual organ model,” as used herein, refers to a digital replica of physical characteristics of a physical organ. For example, a virtual organ model may be a two dimensional or three-dimensional model of a physical organ, such as a heart, liver, lung, or pancreas. However, as used herein, “physical organ” may also be more rigid bodily features, such as bones, muscles, and cartilage.

Test system 200 also includes medical device 230, which may be coupled to the computing device 230. Medical device 230 may include one or more medical devices and/or tools used for medical testing and diagnosis. In some embodiments, medical device 230 may include (but is not limited to) a digital stethoscope, a digital thermometer, a blood pressure monitor, a pulse oximeter, a glucose meter, an ECG monitor, an ultrasound machine, a spirometer, a fetal Doppler device, an x-ray device, an MRI machine, and so on.

Medical device 230 may collect measurements relating to a physical organ. For instance, a digital stethoscope may collect heart rate data and/or heart rhythm pattern data, while an ECG monitor may collect heartbeat voltage data, heart rhythm pattern data, and/or heart polarization data.

Test system 200 may also include an application server 240, which may be coupled to the computing device 230. In some embodiments, the application server 240 may be a cloud server, as illustrated in FIG. 2. A cloud server, as used herein, refers to a computing device that may be housed at various remote locations and is accessed using a networking protocol, such as those described above with reference to network interface 106. In some embodiments, the application server 240 is located on the same local network as the computing device 210.

Application server 240 may include data storage 242. As a possible example, data storage 242 may include any form of database, such as a structured query language (SQL) database or a No-SQL database (e.g., MongoDB). Various types of data structures may store the information in such a database, including but not limited to files, tables, arrays, lists, trees, and tuples. In some embodiments, data storage 242 may store one or more virtual organ models. Furthermore, any databases in data storage 242 may be monolithic or distributed across multiple physical devices.

Application server 240 may be configured to transmit data to and receive data from data storage 242. This transmission and retrieval may take the form of SQL queries or other types of database queries, and the output of such queries, respectively. Additional text, images, video, and/or audio may be included as well. Furthermore, application server 240 may organize the received data into web page or web application representations. Such a representation may take the form of a markup language with or without embedded executable code, such as Hypertext Markup Language (HTML), Extensible Markup Language (XML), JavaScript Object Notation (JSON), or some other standardized or proprietary format.

Application server 240 may also include model training 244. Model training 244 may involve the creation, training, and deployment of one or more machine learning (ML) and/or artificial intelligence (AI) models 246. Such models may be configured to run on the computing device 210 as an application 104C. As noted above, applications 104C may be one or more user-space software programs, as well as any software libraries used by these programs, that are configured to run on a computing device such as the computing device 210.

The models 246 may be configured to recognize, from an image and/or image stream (i.e. image representations), a body pose and subsequently identify one or more predetermined body points. For instance, the models 246 may be trained using a supervised learning process as part of model training 244. Supervised learning is called “supervised” since it trains models on manually labeled information to teach the models to associate certain elements with other data and detect patterns, as contrasted with unsupervised learning which makes use of unlabeled data.

For example, for a model configured to detect the torso of a subject, it may be trained on previous images and image streams containing torsos and that have predetermined body points marked, such as “left shoulder,” “right shoulder,” “left hip,” and/or “right hip,” which generally demarcate the torso of a subject. During this supervised training process, the models 246 learn associations between parts of the images and the predetermined body points, such that when presented with an input image, the models 246 may be able to provide predictions of expected locations of body points and thus the torso area demarcated by such points.

In other words, the models 246 may be trained with a plurality of associations between prior image representations of bodies of organisms and labeled body points within the prior image representations, wherein the labeled body points identify expected locations of physical organs, and wherein the trained machine learning model is arranged to provide predictions of expected locations of body points within an input image representation of a body.

As noted above, the models 246 may be trained during model training 244 on previous examples of images, image streams, and identified predetermined body points. After initial training, this may be repeated with collected image representations in order to improve the recognition accuracy of the models 246. This re-training process is known as “fine-tuning.” In some embodiments, image representations may be transmitted to the application server 240 and stored in data storage 242 and subsequently used by model training 244 to fine-tune the models 246. This fine-tuning process may then use a set of further labeled body points during the fine-tuning process.

In other words, the process may involve receiving further labeled body points associated with the image representation. The further labeled body points identify expected locations of physical organs. The process may then involve training a further trained machine learning model with a one or more associations between the image representation and the further labeled body points. The further trained machine learning model may arranged to provide predictions of expected locations of body points within an input image representation of a body.

As noted above, the application server 240 may be coupled to the computing device 230. This may enable for bidirectional communication between the application server 240 and the computing device 230. For example, a trained model from models 246 may be transmitted to the computing device 230 to run as an application on the computing device, as described above.

### III. Example Processes

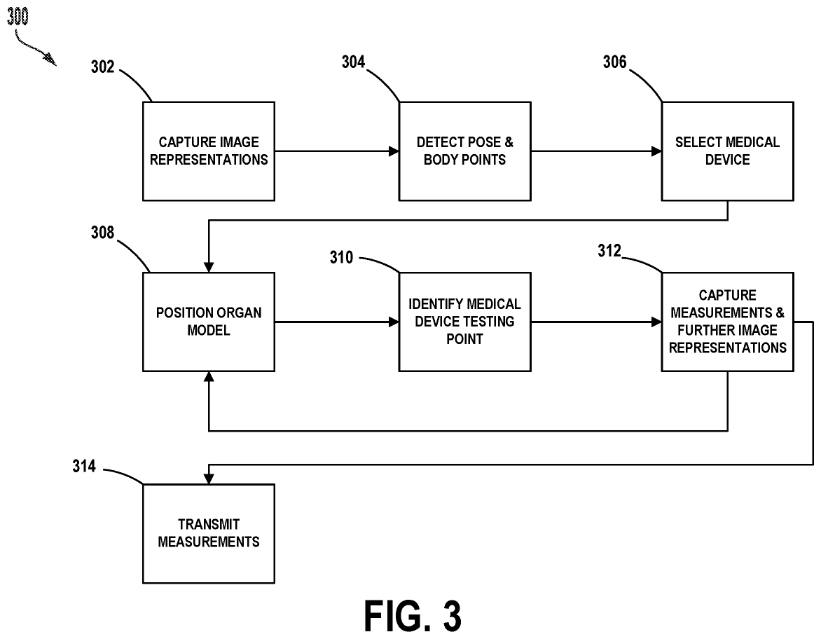

FIG. 3 illustrates an example process 300. Process 300 may operate on or in connection with a test system, such as the test system 200 illustrated in FIG. 2.

Block 302 may involve capturing image data. For example, an imaging device 220 may capture an image representation and subsequently transmit the image representation to the computing device 210. The imaging device 220 may be a camera, an infrared camera, an x-ray device, an ultrasound device, an MRI machine, a LiDAR device, and/or others.

Block 304 may involve detecting a body pose and predetermined body points. As noted above, the body pose refers to specific alignments and positions of a body and/or its parts at a given moment, possibly including orientation, angles, and placements of a head, torso, limbs, and so on. As a part of detecting the body pose, block 304 may involve identifying one or more predetermined body points. This may be performed by a trained machine learning model (much as models 246) based on the captured image data. As described above, once a machine learning model has been trained on the previous examples, it may be able to identify the predetermined body points and thus the body pose within the image representation.

Block 306 may involve selecting medical device. This may occur automatically in response to a medical device, tool, or other equipment being coupled to the mobile device, though in some instances the medical device may be selected by a user in response to a prompt via a graphical interface. In some embodiments, the medical device may include (but is not limited to) one or more of a digital stethoscope, a digital thermometer, a blood pressure monitor, a pulse oximeter, a glucose meter, an ECG monitor, an ultrasound machine, a spirometer, a fetal Doppler device, and so on.

Block 308 may involve positioning a virtual organ model on the image representation. The virtual organ model may be selected automatically based on the medical device of the block 306. For example, if an ECG is selected, a virtual organ model of a heart may be selected for positioning. In some embodiments, the virtual organ model may be selected by a user via a graphical interface from a list of available virtual organ models. Once an appropriate virtual organ model is selected, the virtual organ model is overlaid with the image representation at the expected location of the physical organ according to the detected body pose and predetermined body points and displayed to augment the displayed image representation. For example, a heart model may be overlaid on a subject's torso, and scaled appropriately to fit the subject. In other words, the process may involve increasing or decreasing the size of the heart model, or it may change its orientation according to the detected body pose and/or other characteristics of the body.

Block 310 may involve identifying a medical device testing point. A testing point is a location of where a medical device is to be positioned with respect to the body. Such testing points may vary based upon the medical device being used and the virtual organ model that has been placed. For example, for a digital stethoscope with a heart virtual organ model, a testing point may be placed around the breastbone, while for a blood pressure monitor such as a cuff, a testing point may be placed around the upper arm.

These testing points may then be highlighted in the image representation, which provides real-time instruction to the user how and where to place one or more medical devices and/or parts of medical devices with respect to the expected location of the physical organ.

Block 312 may involve capturing measurements from the medical device (e.g. medical device 230) and capturing further image data (e.g. a stream of image representations) from the imaging device. Thus, the medical information and images of the test taking place are captured together and associated with each other. In some embodiments, the stream of image representations may be time-synchronized with the measurements, which may include the output data from the medical device. In other words, clocks on both the medical device and the mobile device may be synchronized. This is so that data collected by the medical device (i.e. the measurements) are properly associated with the mobile device's stream of image representations. To accomplish this, the measurements may be timestamped according to the synchronized time, such that the results are associated with a specific moment in the stream of image representations. This time synchronization may occur through communication with a time server, for example an implementation of the Network Time Protocol (NTP) and communication with an NTP server. As noted above, the timestamped measurements may also be used to produce or calculate additional information in some embodiments. One example is the PTT (Pulse Transmission Time) in the case of cardiovascular applications, as PTT can be used for measuring the stiffness of blood vessels. As another example, a timestamp may be used to measure the time elapsed during a medical event, e.g. the time elapsed from mitral valve closure in the heart to a pulse in the wrist, which may aid in diagnosing circulatory problems.

In some embodiments, the results and/or stream of image representations may be used to further adjust the virtual organ model and/or its positioning. Thus, the process may return to block 308 to adjust or reposition the model based on the collected results or stream of image representations. In other words, a variation associated with the physical organ may be identified, and the structure of the virtual organ model may be subsequently modified such that the virtual organ model corresponds to the variation associated with the physical organ. For example, a digital stethoscope's heartbeat rate may be used to adjust atrium and ventricle contracting of the heart virtual organ model, and 12-lead ECG results may be used to identify a blockage within the heart and subsequently adjust the structure of the heart virtual organ model.

Block 314 may involve transmitting the measurements and stream of image representations to a server, writing them to memory, or otherwise storing them. Such records, which may be time-synchronized as described above, may be retained for future analysis, though they may also be used to further train the machine-learning models in order to improve the accuracy of the machine-learning models. For instance, the measurements and stream of images from the test system 200 may be transmitted to the application server 240 and stored in data storage 242. Then, this stored information may be used to “fine-tune” the models 246 using model training 244, as described above with relation to FIG. 2. By training models 246 on more examples of body poses, measurements, image streams, and predetermined body points, the models 246 may become more accurate, thus improving the overall functionality of the embodiments herein.

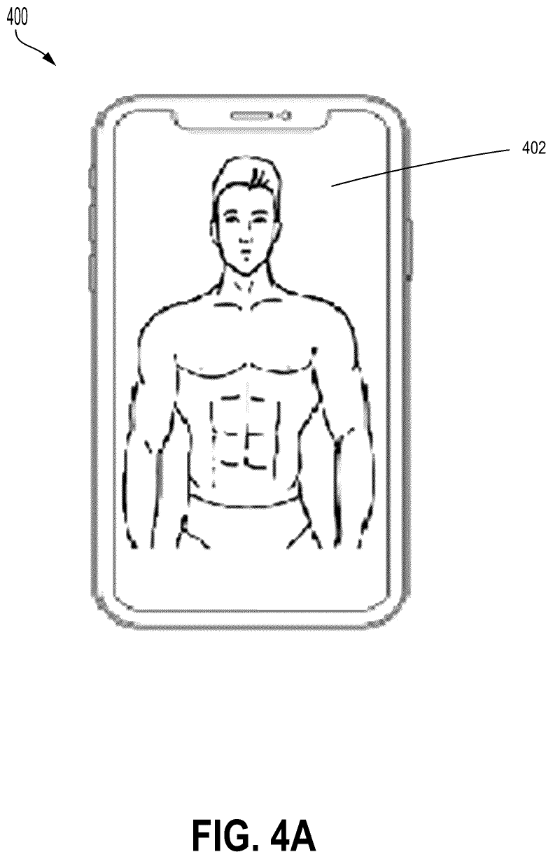

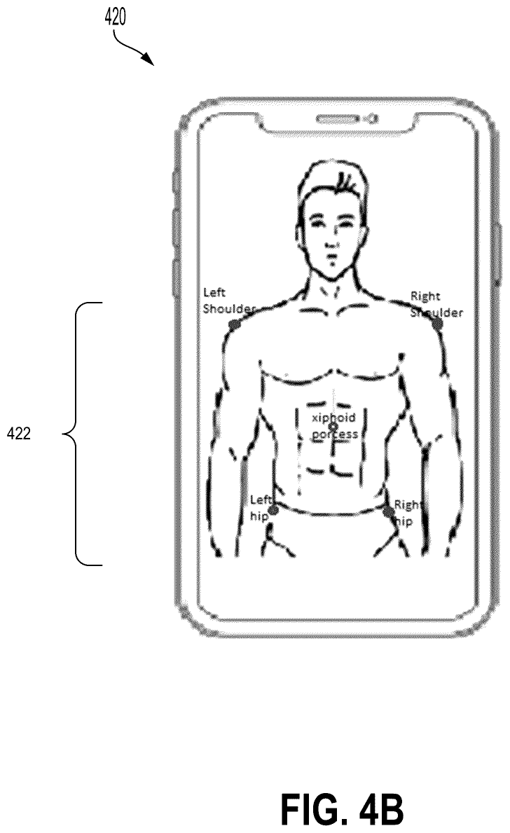

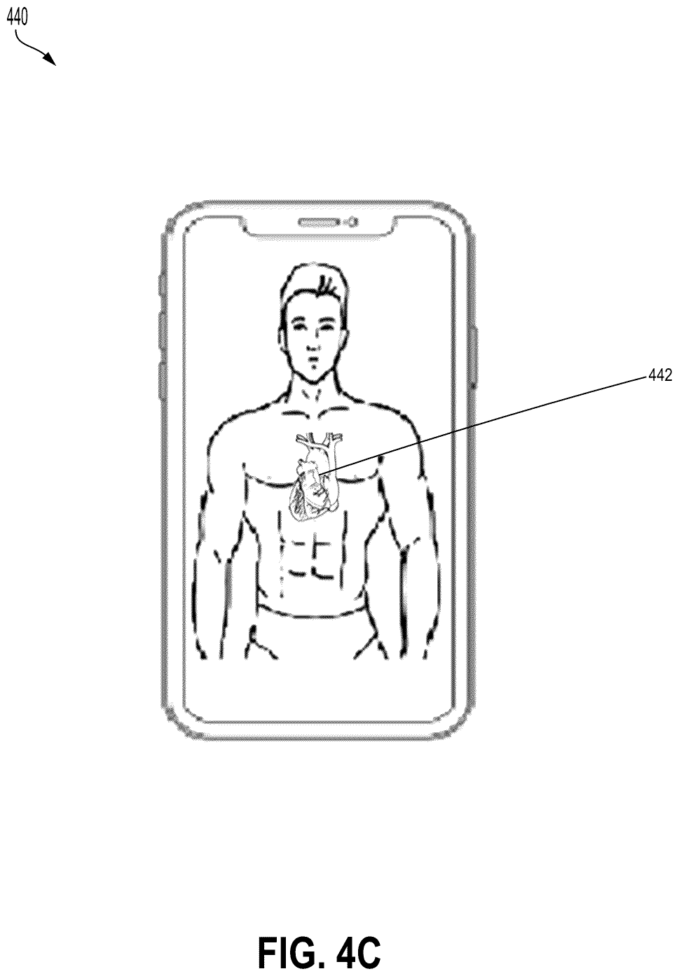

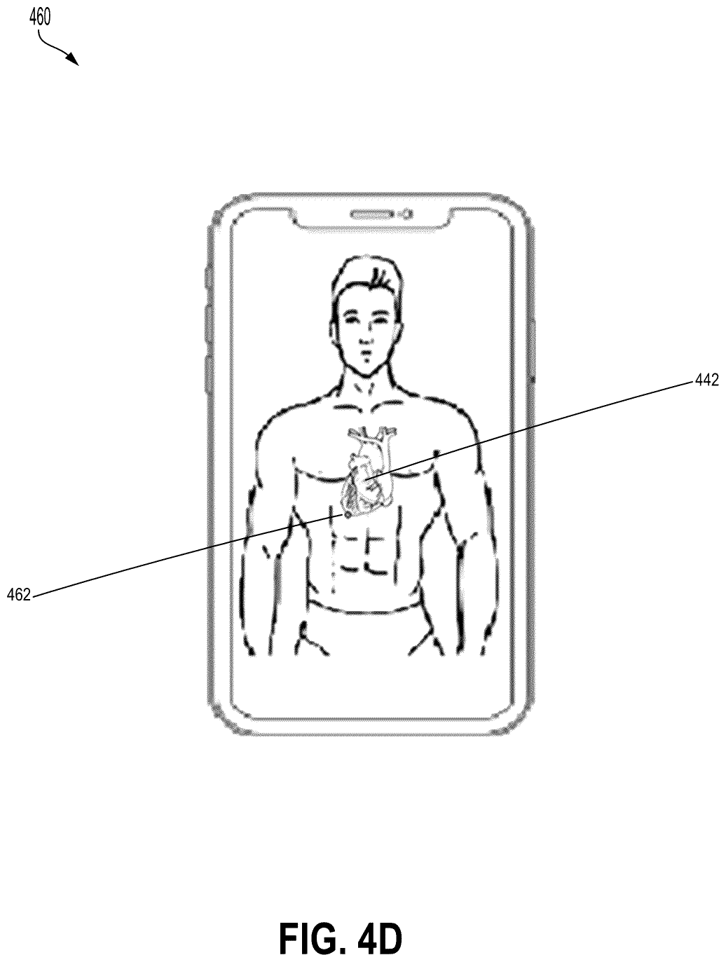

FIGS. 4A-4B depict a specific example of the process 300 operating in connection with a mobile device. Such a mobile device may be a component of an example configuration of the test system 200, and may comprise a computing device 210 and imaging device 220.

FIG. 4A depicts a mobile device in a first state 400. In this example, the mobile device has captured, by an imaging device, an image representation 402. The image representation 402 is thus a representation of the body of an organism. In this example, the image representation 402 depicts the upper body of a male human being.

FIG. 4B illustrates the mobile device in a second state 420. In this example, the mobile device has identified a set of predetermined body points 422, including the right and left shoulders, left and right hips, and xiphoid process. This identification may done by a trained machine learning model running as an application on the mobile device. The trained machine learning model may be received from the application server 240 as a model 246.

In some embodiments, some of the predetermined body points 422 may be more difficult for the mobile device to identify. In such a case, the mobile device may, by way of a graphical interface, prompt a user to locate a specific key body point 422. For example, the xiphoid process, which is a bone and thus not directly locatable through external visual images as used herein.

FIG. 4C illustrates the mobile device in a third state 440. In this example, the mobile device has superimposed a virtual organ model 442 of a heart onto the image representation 402. This virtual image model may have been selected automatically based upon the mobile device's connection or coupling to a medical device or other medical device, but in some embodiments the virtual organ model may be selected manually by a user of the mobile device via a graphical interface. For example, if a digital stethoscope was connected to the mobile device, it may automatically select a heart virtual organ model 442 for positioning onto the image representation 402.

The positioning of the virtual organ model 442 may be based upon the identified predetermined body points, as discussed above with reference to FIG. 4B. In some embodiments, the virtual organ model may be a three-dimensional digital model of an organ. The model size is adjusted to fit the body and the location of the model is calculated relative to the detected predetermined body points.

FIG. 4D illustrates the mobile device in a fourth state 460. In this example, the mobile device has superimposed a testing point 462 onto the image representation 402 along with the virtual organ model 442. The testing point 462 indicates a location for a user to attach or otherwise place a medical device onto the body. For example, testing point 462 may represent a favorable place to position a digital stethoscope in order to listen to the rhythms of the heart.

In some embodiments, the test pointing 462 may be positioned based on the virtual organ model 442 and its positioning, including the type of physical organ. Based on the equipment type, property, and the nature of the physical organ, the mobile device may calculate the proper location of the test point 462 relative to the virtual organ model 442. The testing point 462 may then be highlighted or superimposed over the displayed body image and presented as a real time guide for the user to place the medical device. Additionally, the virtual organ model 442 may not reflect an organ exactly, and thus changing the test point 462 may create differences. Thus, the mobile device may adjust the location of the test point 462 to more accurately measure the true position of the organ in response to data collected by the media device, as described below.

At this point, once a user has attached or otherwise positioned the medical device with respect to the body, it may begin collecting data. In the example of the digital stethoscope, it may record a heartbeat and other heart-related information. Simultaneously, further image data may be collected by the imaging device along with the data from the medical device, thus creating a stream of images related to the test procedure. In response to this information, the virtual organ model 442 and its positioning may be actively adjusted. For example, a digital stethoscope's heartbeat rate may be used to adjust atrium and ventricle contracting of the heart virtual organ model 442, and 12-lead ECG results may be used to adjust the orientation of the heart virtual organ model 442.

The data collected by the medical device (i.e. measurements), along with the stream of images, may be time-synchronized, as described above. The clock of the medical device is synchronized to the mobile device's clock, and may be verified via communication with a time server, for example via an NTP communication. Measurements may then be time stamped, including the stream of images associated with the measurements. This information may be used for further analysis. For example, a timestamp may be used to measure the time elapsed during a medical event, e.g. the time elapsed from mitral valve closure in the heart to a pulse in the wrist, which may aid in diagnosing circulatory problems.

Timestamps in measurements and the stream of images may also be captured to show how measurements are obtained if measurements are presented to other people, including medical professionals. For example, a particular image representation captured at a specific time may be displayed on a graphical interface, and information relating to a particular measurement made within a threshold amount of time from the particular timestamp may also be displayed on the graphical interface. This may allow for analysis tasks (e.g., at this point in the procedure or test, this particular result was obtained).

Additionally, the measurements together with the timestamped stream of images may be stored in the mobile device, for example written into memory 104. In some embodiments, the detected predetermined body points 422, equipment/medical device type, test points 462, measurements, and captured stream of images may be transmitted to a server for storage. This data may then be used for training the machine learning models used herein. For instance, the data may be used to improve the key body point detection and test point estimation accuracy, and thus to further improve the functioning of the system. In some embodiments, the machine learning models trained on the server may be transmitted to the mobile device for use as an application.

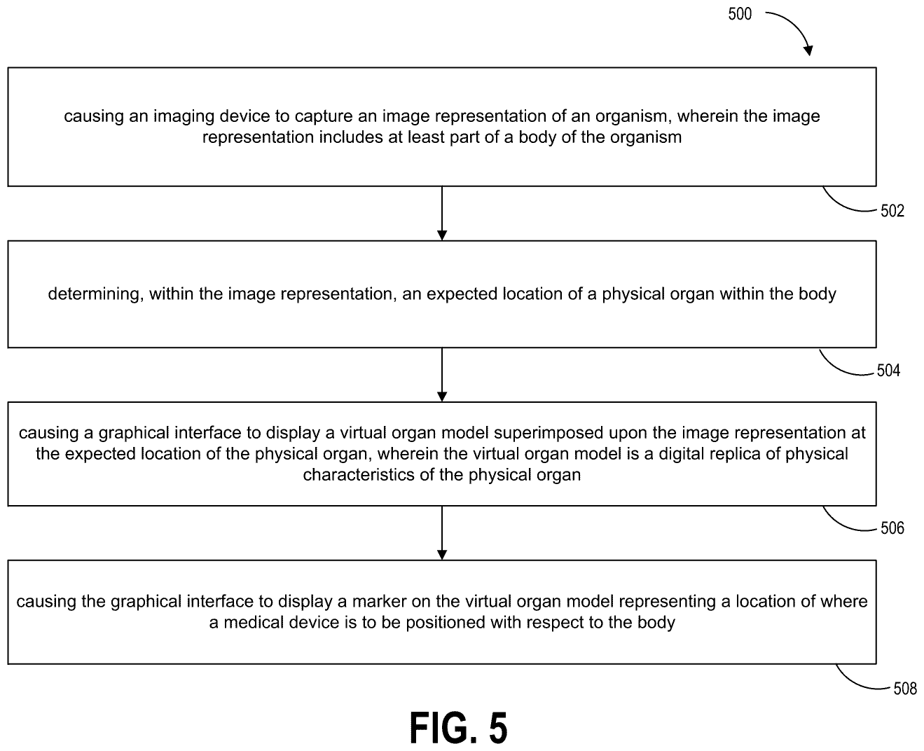

FIG. 5 depicts a flow chart, in accordance with example embodiments. The process 500 illustrated by FIG. 5 may be carried out by a computing device, such as computing device 100, and/or a system, such as test system 200. However, the process can be carried out by other types of devices, systems, or device subsystems. For example, the process could be carried out by a portable computer or mobile device, such as a laptop, a smartphone, or a tablet device.

The embodiments of FIG. 5 may be simplified by the removal of any one or more of the features shown therein. Further, these embodiments may be combined with features, aspects, and/or implementations of any of the previous figures or otherwise described herein.

Block 502 may involve causing an imaging device to capture an image representation of an organism, wherein the image representation includes at least part of a body of the organism.

Block 504 may involve determining, within the image representation, an expected location of a physical organ within the body.

Block 506 may involve causing a graphical interface to display a virtual organ model superimposed upon the image representation at the expected location of the physical organ, wherein the virtual organ model is a digital replica of physical characteristics of the physical organ.

Block 508 may involve causing the graphical interface to display a marker on the virtual organ model representing a location of where a medical device is to be positioned with respect to the body.

In some embodiments, the process 500 may further involve receiving an indication that the medical device has been positioned at the location. In some embodiments, the process 500 may further involve, in response to receiving the indication, causing the imaging device to capture a stream of further image representations of the organism and causing the medical device to obtain measurements relating to the physical organ. In some embodiments, the process 500 may further involve causing at least a portion of the stream of further image representations and a portion of the measurements to be stored in a memory, each with associated timestamps.

In some embodiments, the process 500 may further involve based on the measurements, identifying a variation associated with the physical organ that differs from the virtual organ model. In some embodiments, the process 500 may further involve modifying a structure of the virtual organ model such that the structure of the virtual organ model corresponds to the variation associated with the physical organ.

In some embodiments, the process 500 may further involve displaying, by way of the graphical interface, a particular image representation of the stream of further image representations, wherein the particular image representation was captured at a time specified by a particular timestamp of the associated timestamps. In some embodiments, the process 500 may further involve displaying, by way of the graphical interface, information relating to a particular measurement of the measurements, wherein the particular measurement was made with a threshold amount of time from the particular timestamp.

In some embodiments, determining the expected location of the physical organ within the body may involve based on the image representation, receiving, from a trained machine learning model, respective locations of predetermined body points. In some embodiments, determining the expected location of the physical organ within the body may involve based on the respective locations of the predetermined body points, selecting the expected location of the physical organ.

In some embodiments, the trained machine learning model may have been trained with a plurality of associations between prior image representations of bodies of organisms and labeled body points within the prior image representations. The labeled body points may identify expected locations of physical organs. The trained machine learning model may be arranged to provide predictions of expected locations of body points within an input image representation of a body.

In some embodiments, the process 500 may further involve receiving further labeled body points associated with the image representation, wherein the further labeled body points identify expected locations of physical organs. In some embodiments, the process 500 may further involve training a further trained machine learning model with a one or more associations between the image representation and the further labeled body points, wherein the further trained machine learning model is arranged to provide predictions of expected locations of body points within an input image representation of a body.

In some embodiments, the imaging device may include a camera, an infrared camera, an x-ray device, an ultrasound device, an MRI machine, or a LiDAR device.

In some embodiments, causing the graphical interface to display the virtual organ model superimposed upon the image representation at the expected location of the physical organ may involve selecting, by way of the graphical interface, the medical device. In some embodiments, causing the graphical interface to display the virtual organ model superimposed upon the image representation at the expected location of the physical organ may involve based on the medical device, selecting the virtual organ model.

In some embodiments, the virtual organ model may include a two-dimensional or a three-dimensional digital replica of physical characteristics of the physical organ.

In some embodiments, the medical device may include a digital stethoscope, a digital thermometer, a blood pressure monitor, a pulse oximeter, a glucose meter, an ECG monitor, an ultrasound machine, a spirometer, or a fetal Doppler device.

In some embodiments, the process 500 may be performed in connection with a computing system. The computing system may include at least one processor, as well as memory and program instructions. The program instructions may be stored in the memory, and upon execution by the at least one processor, cause the computing system to perform operations in accordance with process 500.

In some embodiments, the process 500 may be performed in connection with a non-transitory machine-readable medium. The non-transitory machine-readable medium may have stored thereon program instructions that, upon execution by a computing system, cause the computing system to perform operations in accordance with process 500.

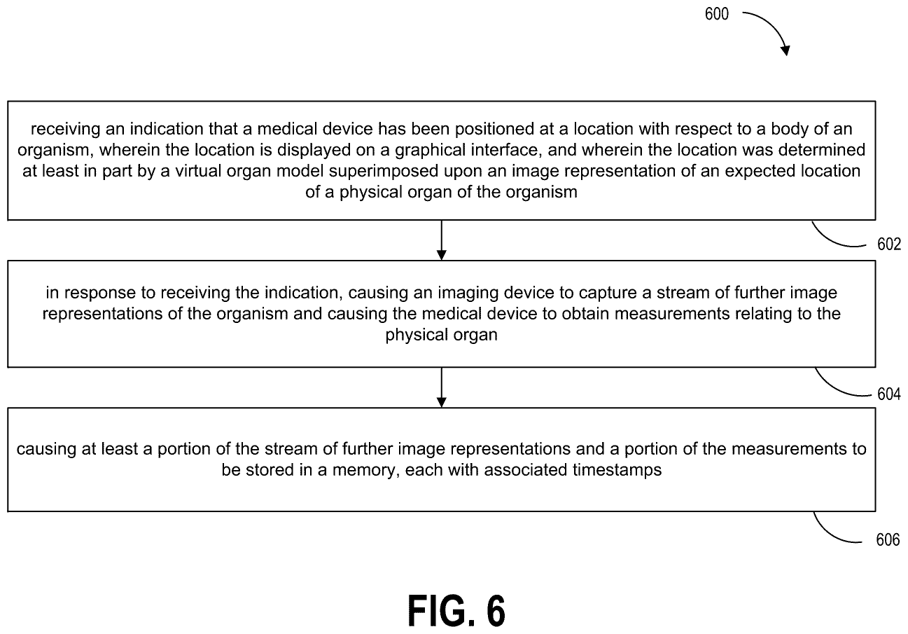

FIG. 6 depicts a flow chart, in accordance with example embodiments. The process 600 illustrated by FIG. 6 may be carried out by a computing device, such as computing device 100, and/or a system, such as test system 200. However, the process can be carried out by other types of devices, systems, or device subsystems. For example, the process could be carried out by a portable computer or mobile device, such as a laptop, a smartphone, or a tablet device.

The embodiments of FIG. 6 may be simplified by the removal of any one or more of the features shown therein. Further, these embodiments may be combined with features, aspects, and/or implementations of any of the previous figures or otherwise described herein.

Block 602 may involve receiving an indication that a medical device has been positioned at a location with respect to a body of an organism, wherein the location is displayed on a graphical interface, and wherein the location was determined at least in part by a virtual organ model superimposed upon an image representation of an expected location of a physical organ of the organism.

Block 604 may involve, in response to receiving the indication, causing an imaging device to capture a stream of further image representations of the organism and causing the medical device to obtain measurements relating to the physical organ.

Block 606 may involve causing at least a portion of the stream of further image representations and a portion of the measurements to be stored in a memory, each with associated timestamps.

In some embodiments, the process 600 may further involve based on the measurements, identifying a variation associated with the physical organ that differs from the virtual organ model. In some embodiments, the process 600 may further involve modifying a structure of the virtual organ model such that the structure of the virtual organ model corresponds to the variation associated with the physical organ.

In some embodiments, the process 600 may further involve displaying, by way of the graphical interface, a particular image representation of the stream of further image representations, wherein the particular image representation was captured at a time specified by a particular timestamp of the associated timestamps. In some embodiments, the process 600 may further involve displaying, by way of the graphical interface, information relating to a particular measurement of the measurements, wherein the particular measurement was made with a threshold amount of time from the particular timestamp.

In some embodiments, the process 600 may further involve receiving further labeled body points associated with the image representation, wherein the further labeled body points identify expected locations of physical organs. In some embodiments, the process 600 may further involve training a further trained machine learning model with a one or more associations between the image representation and the further labeled body points, wherein the further trained machine learning model is arranged to provide predictions of expected locations of body points within an input image representation of a body.

In some embodiments, the process 600 may be performed in connection with a computing system. The computing system may include at least one processor, as well as memory and program instructions. The program instructions may be stored in the memory, and upon execution by the at least one processor, cause the computing system to perform operations in accordance with process 600.

In some embodiments, the process 600 may be performed in connection with a non-transitory machine-readable medium. The non-transitory machine-readable medium may have stored thereon program instructions that, upon execution by a computing system, cause the computing system to perform operations in accordance with process 600.

### IV. CONCLUSION

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, operation, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step, block, or operation that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer-readable medium such as a storage device including RAM, a disk drive, a solid state drive, or another storage medium.

The computer-readable medium can also include non-transitory computer-readable media such as computer-readable media that store data for short periods of time like register memory and processor cache. The computer-readable media can further include non-transitory computer-readable media that store program code and/or data for longer periods of time. Thus, the computer-readable media may include secondary or persistent long term storage, like ROM, optical or magnetic disks, solid state drives, compact-disc read only memory (CD-ROM), for example. The computer-readable media can also be any other volatile or non-volatile storage systems. A computer-readable medium can be considered a computer-readable storage medium, for example, or a tangible storage device.

Moreover, a step, block, or operation that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. A method comprising:
causing an imaging device to capture an image representation of an organism, wherein the image representation includes at least part of a body of the organism;
determining, within the image representation, an expected location of a physical organ within the body;
causing a graphical interface to display a virtual organ model superimposed upon the image representation at the expected location of the physical organ, wherein the virtual organ model is a digital replica of physical characteristics of the physical organ; and
causing the graphical interface to display a marker on the virtual organ model representing a location of where a medical device is to be positioned with respect to the body.

2. The method of claim 1, further comprising:
receiving an indication that the medical device has been positioned at the location;
in response to receiving the indication, causing the imaging device to capture a stream of further image representations of the organism and causing the medical device to obtain measurements relating to the physical organ; and
causing at least a portion of the stream of further image representations and a portion of the measurements to be stored in a memory, each with associated timestamps.

3. The method of claim 2, further comprising:
based on the measurements, identifying a variation associated with the physical organ that differs from the virtual organ model; and
modifying a structure of the virtual organ model such that the structure of the virtual organ model corresponds to the variation associated with the physical organ.

4. The method of claim 2, further comprising:
displaying, by way of the graphical interface, a particular image representation of the stream of further image representations, wherein the particular image representation was captured at a time specified by a particular timestamp of the associated timestamps; and
displaying, by way of the graphical interface, information relating to a particular measurement of the measurements, wherein the particular measurement was made with a threshold amount of time from the particular timestamp.

5. The method of claim 1, wherein determining the expected location of the physical organ within the body comprises:
based on the image representation, receiving, from a trained machine learning model, respective locations of predetermined body points; and
based on the respective locations of the predetermined body points, selecting the expected location of the physical organ.

6. The method of claim 5, wherein the trained machine learning model has been trained with a plurality of associations between prior image representations of bodies of organisms and labeled body points within the prior image representations, wherein the labeled body points identify expected locations of physical organs, and wherein the trained machine learning model is arranged to provide predictions of expected locations of body points within an input image representation of a body.

7. The method of claim 5, further comprising:
receiving further labeled body points associated with the image representation, wherein the further labeled body points identify expected locations of physical organs; and
training a further trained machine learning model with a one or more associations between the image representation and the further labeled body points, wherein the further trained machine learning model is arranged to provide predictions of expected locations of body points within an input image representation of a body.

8. The method of claim 1, wherein the imaging device comprises a camera, an infrared camera, an x-ray device, an ultrasound device, a magnetic resonance imaging (MRI) machine, or a light detection and ranging (LiDAR) device.

9. The method of claim 1, wherein causing the graphical interface to display the virtual organ model superimposed upon the image representation at the expected location of the physical organ comprises:
selecting, by way of the graphical interface, the medical device; and
based on the medical device, selecting the virtual organ model.

10. The method of claim 1, wherein the virtual organ model comprises a two-dimensional or a three-dimensional digital replica of physical characteristics of the physical organ.

11. The method of claim 1, wherein the medical device comprises a digital stethoscope, a digital thermometer, a blood pressure monitor, a pulse oximeter, a glucose meter, an electrocardiogramonitor, an ultrasound machine, a spirometer, or a fetal Doppler device.

12. A method comprising:
receiving an indication that a medical device has been positioned at a location with respect to a body of an organism, wherein the location is displayed on a graphical interface, and wherein the location was determined at least in part by a virtual organ model superimposed upon an image representation of an expected location of a physical organ of the organism;
in response to receiving the indication, causing an imaging device to capture a stream of further image representations of the organism and causing the medical device to obtain measurements relating to the physical organ; and
causing at least a portion of the stream of further image representations and a portion of the measurements to be stored in a memory, each with associated timestamps.

13. The method of claim 12, further comprising:
based on the measurements, identifying a variation associated with the physical organ that differs from the virtual organ model; and
modifying a structure of the virtual organ model such that the structure of the virtual organ model corresponds to the variation associated with the physical organ.

14. The method of claim 12, further comprising:
displaying, by way of the graphical interface, a particular image representation of the stream of further image representations, wherein the particular image representation was captured at a time specified by a particular timestamp of the associated timestamps; and
displaying, by way of the graphical interface, information relating to a particular measurement of the measurements, wherein the particular measurement was made with a threshold amount of time from the particular timestamp.

15. The method of claim 12, further comprising:
receiving further labeled body points associated with the image representation, wherein the further labeled body points identify expected locations of physical organs; and
training a further trained machine learning model with a one or more associations between the image representation and the further labeled body points, wherein the further trained machine learning model is arranged to provide predictions of expected locations of body points within an input image representation of a body.

16. A system comprising:
an imaging device;
a medical device; and
a computing device comprising one or more processors, memory, and program instructions, stored in the memory, that upon execution by the one or more processors cause the system to perform operations comprising:
causing the imaging device to capture an image representation of an organism, wherein the image representation includes at least part of a body of the organism;
determining, within the image representation, an expected location of a physical organ within the body;
causing a graphical interface to display a virtual organ model superimposed upon the image representation at the expected location of the physical organ, wherein the virtual organ model is a digital replica of physical characteristics of the physical organ; and
causing the graphical interface to display a marker on the virtual organ model representing a location of where the medical device is to be positioned with respect to the body.

17. The system of claim 16, wherein the operations further comprise:
receiving an indication that the medical device has been positioned at the location;
in response to receiving the indication, causing the imaging device to capture a stream of further image representations of the organism and causing the medical device to obtain measurements relating to the physical organ; and
causing at least a portion of the stream of further image representations and a portion of the measurements to be stored in the memory, each with associated timestamps.

18. The system of claim 17, wherein the operations further comprise:
based on the measurements, identifying a variation associated with the physical organ that differs from the virtual organ model; and
modifying a structure of the virtual organ model such that the structure of the virtual organ model corresponds to the variation associated with the physical organ.

19. The system of claim 16, wherein determining the expected location of the physical organ within the body comprises:
based on the image representation, receiving, from a trained machine learning model, respective locations of predetermined body points; and
based on the respective locations of the predetermined body points, selecting the expected location of the physical organ.

20. The system of claim 19, wherein the trained machine learning model has been trained with a plurality of associations between prior image representations of bodies and labeled body points within the prior image representations, wherein the labeled body points identify expected locations of physical organs, and wherein the trained machine learning model is arranged to provide predictions of expected locations of body points within an input image representation of a body.
